SEQUENCE LISTING (28 sequences)

SEQ ID NO: 1 - protein, 8 aa, Unknown
  Description of Unknown Angiotensin II peptide
DRVYIHPF

SEQ ID NO: 2 - protein, 8 aa, Artificial Sequence
  Description of Artificial Sequence Synthetic peptide
DRVYVHPF

SEQ ID NO: 3 - protein, 8 aa, Artificial Sequence
  Description of Artificial Sequence Synthetic peptide
NRVFIHPF

SEQ ID NO: 4 - protein, 9 aa, Artificial Sequence
  Description of Artificial Sequence Synthetic peptide
NRVYYVHPF

SEQ ID NO: 5 - protein, 8 aa, Artificial Sequence
  Description of Artificial Sequence Synthetic peptide
NRVYIHPI

SEQ ID NO: 6 - protein, 8 aa, Artificial Sequence
  Description of Artificial Sequence Synthetic peptide
NRVYIHPA

SEQ ID NO: 7 - protein, 8 aa, Artificial Sequence
  Description of Artificial Sequence Synthetic peptide
NRVYIHPF

SEQ ID NO: 8 - protein, 7 aa, Unknown
  Description of Unknown Angiotensin III peptide
RVYIHPF

SEQ ID NO: 9 - protein, 7 aa, Artificial Sequence
  Description of Artificial Sequence Synthetic peptide
RVYVHPF

SEQ ID NO: 10 - protein, 7 aa, Artificial Sequence
  Description of Artificial Sequence Synthetic peptide
RVFIHPF

SEQ ID NO: 11 - protein, 7 aa, Artificial Sequence
  Description of Artificial Sequence Synthetic peptide
RVYIHPA

SEQ ID NO: 12 - protein, 7 aa, Artificial Sequence
  Description of Artificial Sequence Synthetic peptide
RVYIHPF

SEQ ID NO: 13 - protein, 6 aa, Unknown
  Description of Unknown Angiotensin IV peptide
VYIHPF

SEQ ID NO: 14 - protein, 6 aa, Artificial Sequence
  Description of Artificial Sequence Synthetic peptide
VYVHPF

SEQ ID NO: 15 - protein, 6 aa, Artificial Sequence
  Description of Artificial Sequence Synthetic peptide
VFIHPF

SEQ ID NO: 16 - protein, 6 aa, Artificial Sequence
  Description of Artificial Sequence Synthetic peptide
VYIHPA

SEQ ID NO: 17 - protein, 6 aa, Artificial Sequence
  Description of Artificial Sequence Synthetic peptide
VYIHPF

SEQ ID NO: 18 - protein, 10 aa, Unknown
  Description of Unknown Angiotensin I peptide
DRVYIHPFHL

SEQ ID NO: 19 - protein, 10 aa, Artificial Sequence
  Description of Artificial Sequence Synthetic peptide
DRVYVHPFHL

SEQ ID NO: 20 - protein, 10 aa, Artificial Sequence
  Description of Artificial Sequence Synthetic peptide
NRVFIHPFHL

SEQ ID NO: 21 - protein, 9 aa, Artificial Sequence
  Description of Artificial Sequence Synthetic peptide
RVYIHPFHL

SEQ ID NO: 22 - protein, 8 aa, Artificial Sequence
  Description of Artificial Sequence Synthetic peptide
VYIHPFHL

SEQ ID NO: 23 - protein, 11 aa, Artificial Sequence
  Description of Artificial Sequence Synthetic peptide
NRVYYVHPFHL

SEQ ID NO: 24 - protein, 10 aa, Artificial Sequence
  Description of Artificial Sequence Synthetic peptide
NRVYIHPIHL

SEQ ID NO: 25 - protein, 10 aa, Artificial Sequence
  Description of Artificial Sequence Synthetic peptide
NRVYIHPAHL

SEQ ID NO: 26 - protein, 10 aa, Artificial Sequence
  Description of Artificial Sequence Synthetic peptide
NRVYIHPFHL

SEQ ID NO: 27 - protein, 485 aa, Homo sapiens [sequence omitted]

SEQ ID NO: 28 - protein, 12 aa, Artificial Sequence
  Description of Artificial Sequence Synthetic peptide
DRVYIHPFHLVI

(19) United States
(12) Patent Application Publication
(10) Pub. No.: US 2017/0196931 A1
(43) Pub. Date: Jul. 13, 2017

(54) Methods of Administering Vasopressors

(71) Applicant: Lakhmir Chawla, San Diego, CA (US)

(72) Inventor: Lakhmir Chawla, San Diego, CA (US)

(21) Appl. No.: 15/400,283

(22) Filed: Jan. 6, 2017

Related U.S. Application Data
(60) Provisional application No. 62/347,259, filed on Jun. 8, 2016.
(60) Provisional application No. 62/276,164, filed on Jan. 7, 2016.

(51) Int. Cl. A61K 38/08; A61K 9/00

(52) U.S. Cl. CPC A61K 38/085; A61K 9/0019

(57) ABSTRACT

The present disclosure relates to the use of angiotensinogen, angiotensin I, angiotensin I, angiotensin III, and/or angiotensin IV in therapeutic methods for the treatment of hypotension, such as catecholamine-resistant hypotension.

### RELATED APPLICATIONS

This application claims the benefit of priority to U.S. Provisional Patent Application Ser. No. 62/347,259, filed Jun. 8, 2016, and U.S. Provisional Patent Application Ser. No. 62/276,164, filed on Jan. 7, 2016, each of which are herein incorporated by reference in their entireties.

### BACKGROUND OF THE INVENTION

Hypotension, if uncorrected, is life-threatening and occurs as the result of various underlying conditions such as trauma, septic shock or drug reactions. The first line of treatment is intravenous fluids and if this fails to correct the hypotension then vasopressors are deployed. The first line vasopressor is a catecholamine infusion. Catecholamines are amines derived from the amino acid tyrosine, and they include epinephrine (adrenaline), norepinephrine (noradrenaline), phenylephrine, and dopamine, which act as both hormones and neurotransmitters that increase blood pressure. While largely effective at treating hypotension, some patients fail to respond to adequate doses and are defined as catecholamine-resistant. These patients frequently have a high mortality and no acceptable alternatives.

The use of high doses of catecholamines in patients with severe hypotension is associated with poor outcomes. For example, the in-patient, 90-day mortality rate is 50-93% for patients who require norepinephrine as a vasopressor at doses that exceed 0.1 μg/kg/min, and 94% of patients who require norepinephrine at doses above 100 μg/min die.

Thus, alternate methods of regulating blood pressure in patients with catecholamine-resistant hypotension are needed.

### SUMMARY OF THE INVENTION

Angiotensinogen, Angiotensin I, Angiotensin II, angiotensin III, and angiotensin IV are hormones naturally produced by the body that regulate blood pressure via vasoconstriction and sodium reabsorption. Angiotensinogen is a polypeptide produced in the liver that is converted into angiotensin I by renin. Subsequently, angiotensin I may be cleaved and converted to angiotensin II by angiotensin converting enzyme (ACE). Angiotensin II is converted to angiotensin III through the removal of an N-terminal aspartate of angiotensin II by aminopeptidase A (APA). Angiotensin III is converted to angiotensin IV by the removal of an N-terminal arginine by aminopeptidase N. The hemodynamic effects of angiotensin II administration have been the subject of numerous clinical studies, demonstrating significant effects on systemic and renal blood flow.

In some embodiments, the methods disclosed herein may comprise treating hypotension by administering an angiotensin therapeutic agent to a patient, measuring the mean arterial pressure of the patient, and titrating a vasopressor and/or the angiotensin therapeutic agent to a change in the mean arterial pressure of the patient. The angiotensin therapeutic agent may be, for example, angiotensinogen, angiotensin I, angiotensin II, angiotensin III, angiotensin IV, or a peptide or protein comprising the sequence set forth in any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, or SEQ ID NO:28.

In some aspects, the invention relates to methods of treating hypotension, such as catecholamine-resistant hypotension, in a patient in need thereof, comprising administering to the patient a composition comprising an angiotensin therapeutic agent. The term “catecholamine-resistant hypotension” as used herein refers to patients who require more than 15 μg/kg/min of dopamine, 0.1 μg/kg/min norepinephrine, or 0.1 μg/kg/min epinephrine as a vasopressor. Dopamine, norepinephrine, and epinephrine may be administered at rates higher than 15 μg/kg/min, 0.1 μg/kg/min, or 0.1 μg/kg/min, respectively, but elevated rates correlate with increased mortality.

In some embodiments, the invention relates to methods of treating hypotension in a human patient receiving a vasopressor and having an initial mean arterial pressure, comprising: administering to the patient a composition comprising an angiotensin therapeutic agent; after a period of time, measuring the mean arterial pressure of the patient; and, if the measured mean arterial pressure is at or above 75 mm Hg, decreasing the rate at which a vasopressor is administered to the patient. In certain such embodiments, if the measured mean arterial pressure is below 75 mm Hg, the method comprises increasing the rate of administration of the angiotensin therapeutic agent.

The term “mean arterial pressure” or “MAP” refers to the average arterial pressure during a single cardiac cycle.

The term “vasopressor,” as used herein, includes catecholamines such as dopamine, norepinephrine, phenylephrine, and epinephrine and their prodrugs, structural analogs, or derivatives that induce similar physiological effects in humans. The term “catecholamine,” as used herein, refers to dopamine, norepinephrine, phenylephrine, and epinephrine and their prodrugs, structural analogs, or derivatives that induce similar physiological effects in humans, e.g., raise mean arterial pressure in healthy human subjects. Vasopressors also include vasopressin and analogs thereof, such as terlipressin, argipressin, desmopressin, felypressin, lypressin, and ornipressin. A vasopressor may be dobutamine or midodrine. In some embodiments, a method comprises administering two or more of an angiotensin therapeutic agent, a catecholamine, vasopressin, a vasopressin analog, dobutamine, and midodrine to a patient. For example, a method may comprise administering angiotensin II, a catecholamine, and either vasopressin or a vasopressin analog to a patient. A method may comprise administering an angiotensin therapeutic agent, a catecholamine, and vasopressin to a patient.

In some embodiments, the invention relates to methods of treating hypotension in a human patient receiving a vasopressor, e.g., a catecholamine, and having an initial mean arterial pressure, comprising: administering to the patient a composition comprising an angiotensin therapeutic agent; after a period of time, measuring the mean arterial pressure of the patient; and, if the measured mean arterial pressure is at or above a predetermined threshold value (e.g., 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 mm Hg), decreasing the rate at which vasopressor is administered to the patient. In certain such embodiments, if the measured mean arterial pressure is below the predetermined threshold value, the method comprises increasing the rate of administration of the angiotensin therapeutic agent.

In some embodiments, the invention relates to methods of treating hypotension in a human patient receiving a vasopressor and having an initial mean arterial pressure, comprising: administering to the patient a composition comprising an angiotensin therapeutic agent; after a period of time, measuring the mean arterial pressure of the patient; and, if the measured mean arterial pressure is at least 10 mm Hg higher than the initial mean arterial pressure, decreasing the rate at which vasopressor is administered to the patient. In certain such embodiments, if the measured mean arterial pressure is less than 10 mm Hg higher than the initial mean arterial pressure, the method comprises increasing the rate of administration of the angiotensin therapeutic agent.

In some embodiments, the invention relates to methods of treating hypotension in a human patient receiving a vasopressor and having an initial mean arterial pressure, comprising: administering to the patient a composition comprising the angiotensin therapeutic agent; after a period of time, measuring the mean arterial pressure of the patient; and, if the measured mean arterial pressure is higher (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45 mm Hg higher) than the initial mean arterial pressure, decreasing the rate at which vasopressor is administered to the patient. In certain such embodiments, if the measured mean arterial pressure is at or below the initial mean arterial pressure, the method comprises increasing the rate of administration of the angiotensin therapeutic agent.

Those of skill in the art will recognize that in the context of the present invention, anti-hypotensive therapeutics can be administered in any suitable way, but are typically administered by continuous infusion. Accordingly, increasing or decreasing a rate of administration can be accomplished by changing the rate of flow of an intravenous drip, changing the concentration of the agent in an intravenous drip, etc. However, the manner in which the rate of administration is changed will depend on the mode of administration of the therapeutic. Where the therapeutic is administered transmucosally or transdermally, the rate may be increased by changing to a higher-release-rate patch or transdermal composition for example. Where the therapeutic is administered orally, the rate may be increased by switching to a higher-dose form, administering additional doses, or administering controlled-release dosage forms with a higher rate of release, for example. Where the therapeutic is administered by inhalation, the rate may be increased by administering additional boluses, a more concentrated bolus, or a faster-release bolus, for example. Other modes of administration (via subcutaneous injection pump, suppository, etc.) can be modulated in analogous fashions, and decreasing the rate of administration can be accomplished by doing the opposite of an action that would increase the rate of administration of the therapeutic.

An angiotensin therapeutic agent may be particularly useful for patients who require potentially harmful doses of vasopressors. Thus, in some embodiments, the invention relates to methods of treating hypotension, wherein, prior to administering the composition, the patient is receiving dopamine, dobutamine, norepinephrine, epinephrine, phenylephrine, terlipressin, vasopressin, or midodrine as a vasopressor.

In some embodiments, the invention relates to methods of treating hypotension wherein the patient has a cardiovascular sequential organ failure assessment score (“SOFA score”) of 1 or greater prior to administering an angiotensin therapeutic agent. For example, a patient may have a cardiovascular SOFA score of 1, 2, 3, or 4. In some embodiments, the patient has a cardiovascular SOFA score of 2, 3, or 4. In other embodiments, the patient has a cardiovascular SOFA score of 3 or 4. In some embodiments, the patient has a cardiovascular SOFA score of 4 prior to initiation of therapy with the angiotensin therapeutic agent.

In some embodiments, the patient is receiving at least 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 μg/kg/min of norepinephrine prior to administration of an angiotensin therapeutic agent. For example, prior to administering the composition, the patient may be receiving at least 0.1 μg/kg/min of norepinephrine. In other embodiments, the patient may be receiving at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg/min of norepinephrine prior to administering an angiotensin therapeutic agent.

Alternatively, hypotension may be treated with epinephrine. Thus, in some embodiments, the patient may be receiving at least 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 μg/kg/min of epinephrine prior to administering an angiotensin therapeutic agent. For example, prior to administering the composition, the patient may be receiving at least 0.1 μg/kg/min of epinephrine. In other embodiments, the patient may be receiving at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg/min of epinephrine prior to administering an angiotensin therapeutic agent.

Alternatively, hypotension may be treated with dopamine. Thus, in some embodiments, the patient may be receiving at least 5, 6, 7, 8, 9, 10, 11, 12, 13, 14, 15, 16, 17, 18, 19, 20, 21, 22, 23, 24, or 25 μg/kg/min of dopamine prior to administering an angiotensin therapeutic agent. For example, prior to administering the composition, the patient may be receiving at least 5 μg/kg/min of dopamine. In other embodiments, the patient may be receiving at least 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, or 2500 μg/min of dopamine prior to administering an angiotensin therapeutic agent.

Alternatively, hypotension may be treated with vasopressin. Thus, in some embodiments, the patient may be receiving at least 1, 2, 3, 4, 5, 6, 7, 8, 9, 10, 11, 12, 13, 14, 15, 16, 17, 18, 19, 20, 21, 22, 23, 24, or 25 mU/kg/min vasopressin. In some embodiments, the patient may be receiving at least 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.25, 0.30, 0.40, 0.50, 0.60, 0.70, 0.80, 0.90, 1.0, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5.0 U/min vasopressin prior to administering an angiotensin therapeutic agent. For example, prior to administering the composition, the patient may be receiving at least 0.01 U/min of vasopressin.

The mean arterial pressure of the patient may be monitored to titrate the angiotensin therapeutic agent and/or the vasopressor. For example, the mean arterial pressure of the patient may be monitored with an indwelling arterial line or by other suitable methods. In some embodiments, an initial mean arterial pressure is measured prior to administering the composition, the composition is administered, and, after a period of time, an additional mean arterial pressure is measured. The period of time may be, for example, about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100, 105, 110, 115, 120, 135, 150, 165, 180, 195, 210, 225, or 240 minutes, or about 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24, 25, 26, 27, 28, 29, 30, 31, 32, 33, 34, 35, 36, 37, 38, 39, 40, 41, 42, 43, 44, 45, 46, 47, or 48 hours or longer. Preferably, the period of time is less than two hours, most preferably about one hour or less.

In certain embodiments, if the measured mean arterial pressure meets or exceeds a target value, then the rate at which vasopressor is administered is decreased. The target value may be, for example, 60, 61, 62, 63, 64, 65, 66, 67, 68, 69, 70, 71, 72, 73, 74, 75, 76, 77, 78, 79, or 80 mm Hg. In certain preferred embodiments, if the measured mean arterial pressure is at or above 75 mm Hg, then the rate at which vasopressor is administered is decreased.

In other embodiments, if the difference between the measured mean arterial pressure and the initial mean arterial pressure meets or exceeds a target value, then the rate at which vasopressor is administered is decreased. The target value may be, for example, 1, 2, 3, 4, 5, 6, 7, 8, 9, 10, 11, 12, 13, 14, 15, 16, 17, 18, 19, 20, 21, 22, 23, 24, or 25 mm Hg. In certain preferred embodiments, if the measured mean arterial pressure is at least 10 mm Hg higher than the initial mean arterial pressure, then the rate at which vasopressor is administered is decreased.

The mean arterial pressure may be measured more than once; for example, the mean arterial pressure may be measured 1, 2, 3, 4, 5, 6, 7, 8, 9, 10, or more times, or even continuously or substantially continuously. The rate at which vasopressor is administered may be decreased in response to each measurement (or the rate of the angiotensin therapeutic agent, or both), depending on whether the measured mean arterial pressure meets or exceeds a target value. Similarly, the rate at which a vasopressor is administered may be increased after a measurement (or the rate of the angiotensin therapeutic agent may be increased, or both), if the measured mean arterial pressure is less than a target value. Similarly, the rate at which vasopressor is administered may be decreased after each measurement (or the rate of the angiotensin therapeutic agent may be decreased, or both), depending on whether the difference between the measured mean arterial pressure and the initial mean arterial pressure is less than a target value. Similarly, the rate at which vasopressor is administered may be increased after a measurement (or the rate of the angiotensin therapeutic agent may be increased, or both), if the difference between the measured mean arterial pressure and the initial mean arterial pressure is less than a target value.

In some embodiments, if the mean arterial pressure of the patient is at or above 75 mm Hg, then the rate at which vasopressor is administered to the patient is decreased. In some embodiments, if the mean arterial pressure of the patient is at or above 65, 66, 67, 68, 69, 70, 71, 72, 73, 74, 75, 76, 77, 78, 79, 80, 81, 82, 83, 84, or 85 mm Hg, then the rate at which vasopressor is administered to the patient is decreased. In some embodiments, if the measured mean arterial pressure is at least 10 mm Hg higher than the initial mean arterial pressure, then the rate at which vasopressor is administered to the patient is decreased. In some embodiments, if the measured mean arterial pressure is at least 1, 2, 3, 4, 5, 6, 7, 8, 9, 10, 11, 12, 13, 14, 15, 16, 17, 18, 19, 20, 21, 22, 23, 24, or 25 mm Hg higher than the initial mean arterial pressure, then the rate at which vasopressor is administered to the patient is decreased. In certain embodiments, the rate at which vasopressor is administered is decreased by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or more. Thus, for example, the rate at which norepinephrine is administered is decreased by at least 15%. In other embodiments, the rate at which the vasopressor is administered is decreased by at least 60%. In some embodiments, the rate at which vasopressor is administered is decreased to 0 μg/kg/min.

The vasopressors may be titrated down while monitoring the mean arterial pressure of a patient, and titration may occur over the course of minutes to hours. Thus, the rate at which a vasopressor is administered may be decreased by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or more over the course of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100, 105, 110, 115, 120, 135, 150, 165, 180, 195, 210, 225, or 240 minutes, or over the course of about 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24, 25, 26, 27, 28, 29, 30, 31, 32, 33, 34, 35, 36, 37, 38, 39, 40, 41, 42, 43, 44, 45, 46, 47, or 48 hours or longer.

In certain embodiments, the angiotensin therapeutic agent is angiotensin I, and angiotensin I is administered at a rate of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 ng/kg/min. For example, in some embodiments, angiotensin I is administered at a rate of about 5 ng/kg/min, 10 ng/kg/min, about 15 ng/kg/min, or about 20 ng/kg/min. Angiotensin II is effective at increasing a patient's MAP at administration rates above 1 ng/kg/min. Thus, in certain embodiments, the angiotensin therapeutic agent is angiotensin II, and angiotensin II is administered at a rate of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 ng/kg/min. For example, in some embodiments, the angiotensin II is administered at a rate of 5 ng/kg/min. In other embodiments, the invention relates to methods of treating hypotension wherein the angiotensin therapeutic agent is angiotensin II, and angiotensin II is administered at a rate of about 20 ng/kg/min. In still other embodiments, angiotensin II is administered at a rate of about 5 ng/kg/min, about 10 ng/kg/min, about 15 ng/kg/min, about 20 ng/kg/min, about 25 ng/kg/min, about 30 ng/kg/min about, 35 ng/kg/min, or about 40 ng/kg/min. In some embodiments, the angiotensin therapeutic agent is angiotensin III, and angiotensin III is administered at a rate of at least 2, 3, 4, 5, 6, 7, 8, 9, 10, 15, 20, 25, 30, 35, 40, 45, 50, 55, 60, 65, 70, 75, 80, 85, 90, 95, or 100 ng/kg/min. For example, angiotensin III may be administered at a rate of about 10 ng/kg/min, about 20 ng/kg/min, or about 50 ng/kg/min. In some embodiments, the angiotensin therapeutic agent is angiotensin IV, and angiotensin IV is administered at a rate of at least 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, or 200 ng/kg/min. For example, angiotensin IV may be administered at a rate of about 20 ng/kg/min, about 50 ng/kg/min, or about 100 ng/kg/min. In some embodiments, the angiotensin therapeutic agent is angiotensinogen, and angiotensinogen is administered at a rate of at least 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, 200, 220, 240, 250, 260, 280, 300, 320, 350, 400, 450, 500, 600, 700, 800, 900, or 1000 ng/kg/min. For example, angiotensinogen may be administered at a rate of about 20 ng/kg/min, about 50 ng/kg/min, about 100 ng/kg/min, about 200 ng/kg/min, about 500 ng/kg/min, or about 1000 ng/kg/min.

Different patients may require higher or lower rates of administration of an angiotensin therapeutic agent to achieve a treatment goal. The rate of administration may be optimized for different patients by administering an angiotensin therapeutic agent and the increasing or decreasing the rate of administration. In some cases, the patient may be administered an initial bolus of an angiotensin therapeutic agent followed by the administration of the angiotensin therapeutic agent at a lower rate. Alternatively, the patient may be administered an angiotensin therapeutic agent at a low rate followed by gradual, elevated rates. Thus, in some embodiments, the method further comprises increasing the rate at which an angiotensin therapeutic agent is administered, and in other embodiments, the method further comprises decreasing the rate at which the angiotensin therapeutic agent is administered.

Angiotensin I or angiotensin II may be administered at an initial rate of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20 ng/kg/min, and the rate may be increased to a final rate of about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 ng/kg/min. For example, angiotensin I or angiotensin II may be administered at an initial rate of about 1 ng/kg/min to about 20 ng/kg/min, and the rate may be increased by about 5 ng/kg/min to about 20 ng/kg/min (e.g., from about 10 ng/kg/min to about 15 ng/kg/min, from about 15 ng/kg/min to about 25 ng/kg/min, from about 20 ng/kg/min to about 25 ng/kg/min, from about 20 ng/kg/min to about 30 ng/kg/min, or from about 20 ng/kg/min to about 40 ng/kg/min).

Similarly, a peptide or protein comprising any one of SEQ ID NO:1-7, SEQ ID NO:18-26, or SEQ ID NO:28 at its N-terminus may be administered at an initial rate of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20 ng/kg/min, and the rate may be increased to a final rate of about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 ng/kg/min. For example, the peptide or protein may be administered at an initial rate of about 1 ng/kg/min to about 20 ng/kg/min, and the rate may be increased by about 5 ng/kg/min to about 20 ng/kg/min (e.g., from about 10 ng/kg/min to about 15 ng/kg/min, from about 15 ng/kg/min to about 25 ng/kg/min, from about 20 ng/kg/min to about 25 ng/kg/min, from about 20 ng/kg/min to about 30 ng/kg/min, or from about 20 ng/kg/min to about 40 ng/kg/min).

Angiotensin I or angiotensin II may be administered at an initial rate of about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 ng/kg/min, and the rate may be decreased to a final rate of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20 ng/kg/min. For example, angiotensin I or angiotensin II may be administered at an initial rate of about 10 ng/kg/min to about 40 ng/kg/min, and the rate may be decreased by about 5 ng/kg/min to about 20 ng/kg/min (e.g., from about 40 ng/kg/min to about 20 ng/kg/min, from about 20 ng/kg/min to about 15 ng/kg/min, from about 20 ng/kg/min to about 10 ng/kg/min, from about 20 ng/kg/min to about 5 ng/kg/min, or from about 10 ng/kg/min to about 5 ng/kg/min).

Similarly, a peptide or protein comprising any one of SEQ ID NO:1-7, SEQ ID NO:18-26, or SEQ ID NO:28 at its N-terminus may be administered at an initial rate of about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 ng/kg/min, and the rate may be decreased to a final rate of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20 ng/kg/min. For example, the peptide or protein may be administered at an initial rate of about 10 ng/kg/min to about 40 ng/kg/min, and the rate may be decreased by about 5 ng/kg/min to about 20 ng/kg/min (e.g., from about 40 ng/kg/min to about 20 ng/kg/min, from about 20 ng/kg/min to about 15 ng/kg/min, from about 20 ng/kg/min to about 10 ng/kg/min, from about 20 ng/kg/min to about 5 ng/kg/min, or from about 10 ng/kg/min to about 5 ng/kg/min).

Angiotensin III or angiotensin IV may be administered at an initial rate of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20 ng/kg/min, and the rate may be increased to a final rate of about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 ng/kg/min. For example, angiotensin III or angiotensin IV may be administered at an initial rate of about 10 ng/kg/min to about 40 ng/kg/min, and the rate may be increased by about 5 ng/kg/min to about 20 ng/kg/min (e.g., from about 10 ng/kg/min to about 15 ng/kg/min, from about 15 ng/kg/min to about 25 ng/kg/min, from about 20 ng/kg/min to about 25 ng/kg/min, from about 20 ng/kg/min to about 30 ng/kg/min, or from about 20 ng/kg/min to about 40 ng/kg/min).

Similarly, a peptide or protein comprising any one of SEQ ID NO:8-17 at its N-terminus may be administered at an initial rate of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20 ng/kg/min, and the rate may be increased to a final rate of about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 ng/kg/min. For example, the peptide or protein may be administered at an initial rate of about 10 ng/kg/min to about 40 ng/kg/min, and the rate may be increased by about 5 ng/kg/min to about 20 ng/kg/min (e.g., from about 10 ng/kg/min to about 15 ng/kg/min, from about 15 ng/kg/min to about 25 ng/kg/min, from about 20 ng/kg/min to about 25 ng/kg/min, from about 20 ng/kg/min to about 30 ng/kg/min, or from about 20 ng/kg/min to about 40 ng/kg/min).

Angiotensin III or angiotensin IV may be administered at an initial rate of about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 ng/kg/min, and the rate may be decreased to a final rate of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20 ng/kg/min. For example, angiotensin I or angiotensin II may be administered at an initial rate of about 20 ng/kg/min to about 40 ng/kg/min, and the rate may be decreased by about 5 ng/kg/min to about 20 ng/kg/min (e.g., from about 40 ng/kg/min to about 20 ng/kg/min, from about 40 ng/kg/min to about 30 ng/kg/min, from about 25 ng/kg/min to about 20 ng/kg/min, from about 20 ng/kg/min to about 15 ng/kg/min, or from about 20 ng/kg/min to about 10 ng/kg/min).

Similarly, a peptide or protein comprising any one of SEQ ID NO:8-17 at its N-terminus may be administered at an initial rate of about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 ng/kg/min, and the rate may be decreased to a final rate of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20 ng/kg/min. For example, the peptide or protein may be administered at an initial rate of about 20 ng/kg/min to about 40 ng/kg/min, and the rate may be decreased by about 5 ng/kg/min to about 20 ng/kg/min (e.g., from about 40 ng/kg/min to about 20 ng/kg/min, from about 40 ng/kg/min to about 30 ng/kg/min, from about 25 ng/kg/min to about 20 ng/kg/min, from about 20 ng/kg/min to about 15 ng/kg/min, or from about 20 ng/kg/min to about 10 ng/kg/min).

An angiotensin therapeutic agent may be titrated while monitoring the MAP of a patient, and titration may occur over the course of minutes to hours. Thus, the rate at which at which an angiotensin therapeutic agent is administered may be increased or decreased over the course of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100, 105, 110, 115, 120, 135, 150, 165, 180, 195, 210, 225, or 240 minutes, or over the course of about 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24, 25, 26, 27, 28, 29, 30, 31, 32, 33, 34, 35, 36, 37, 38, 39, 40, 41, 42, 43, 44, 45, 46, 47, or 48 hours or longer.

An angiotensin therapeutic agent may be administered as long as necessary to maintain a MAP above a target value. Alternatively, an angiotensin therapeutic agent may be administered until the MAP of a patient can be maintained at a lower dose of vasopressor. In some embodiments, the composition is administered until the mean arterial pressure of the patient can be maintained at or above 70 mm Hg with less than 0.1 μg/kg/min norepinephrine, less than 0.1 μg/kg/min epinephrine, or less than 15 μg/kg/min dopamine. In other embodiments, the composition is administered continuously over a period of time selected from less than 6 hours; from 6 hours to 24 hours; or at least 24 hours. In other embodiments, the composition is administered continuously for at least 1-6 days, such as 1-11 days.

The methods disclosed herein can use any suitable form or analog of angiotensin II that exhibits the desired effect of increasing MAP in human subjects. In some embodiments, the angiotensin therapeutic agent has the sequence set forth in SEQ ID NO:1, SEQ ID NO:2, SEQ ID NO:3, SEQ ID NO:4, SEQ ID NO:5, SEQ ID NO:6, or SEQ ID NO:7, which correspond to species of angiotensin II. Preferably, the angiotensin therapeutic agent has the sequence set forth in SEQ ID NO:1, which is the amino acid sequence of human angiotensin II. In some embodiments, the angiotensin therapeutic agent is selected from 5-L-valine angiotensin II; 1-L-asparagine-5-L-valine angiotensin II; 5-L-isoleucine angiotensin II; and 1-L-asparagine-5-L-isoleucine angiotensin II, preferably 5-L-isoleucine angiotensin II (human angiotensin II; 1-L-aspartate-5-L-isoleucine angiotensin II).

The angiotensin therapeutic agent may have the sequence set forth in SEQ ID NO:8, SEQ ID NO:9, SEQ ID NO:10, SEQ ID NO:11, or SEQ ID NO:12, which correspond to species of angiotensin III. Preferably, angiotensin III has the sequence set forth in SEQ ID NO:8, which is the amino acid sequence of human angiotensin III. In some embodiments, the angiotensin III is selected from 4-L-valine angiotensin III and 4-L-isoleucine angiotensin III, preferably 4-L-isoleucine angiotensin III (human angiotensin III).

The angiotensin therapeutic agent may have the sequence set forth in SEQ ID NO:13, SEQ ID NO:14, SEQ ID NO:15, SEQ ID NO:16, or SEQ ID NO:17, which are species of angiotensin IV. Preferably, angiotensin IV has the sequence set forth in SEQ ID NO:13, which corresponds to human angiotensin IV. In some embodiments, the angiotensin IV is selected from 3-1-L-asparagine-5-L-isoleucine angiotensin II-L-valine angiotensin IV and 3-L-isoleucine angiotensin IV, preferably 3-L-isoleucine angiotensin IV (human angiotensin IV).

In some embodiments, the angiotensin therapeutic agent has the sequence set forth in SEQ ID NO:18, SEQ ID NO:19, SEQ ID NO:20, SEQ ID NO:21, SEQ ID NO:22, SEQ ID NO:23, SEQ ID NO:24, SEQ ID NO:25, or SEQ ID NO:26, which correspond to species of angiotensin I. Preferably, the angiotensin therapeutic agent has the sequence set forth in SEQ ID NO:18, which is the amino acid sequence of human angiotensin I. In some embodiments, the angiotensin therapeutic agent is selected from 5-L-valine angiotensin I; 1-L-asparagine-5-L-valine angiotensin I; 5-L-isoleucine angiotensin I; and 1-L-asparagine-5-L-isoleucine angiotensin I, preferably 5-L-isoleucine angiotensin I (human angiotensin I; 1-L-aspartate-5-L-isoleucine angiotensin I).

In some embodiments, the angiotensin therapeutic agent has the amino acid sequence set forth in SEQ ID NO:27, which corresponds to human angiotensinogen. The angiotensin therapeutic agent may be formulated as a pharmaceutically acceptable salt, for example, as an acetate salt. The methods disclosed herein can use any suitable form or analog of angiotensinogen, angiotensin I, angiotensin II, angiotensin III, or angiotensin IV that exhibits the desired effect of increasing MAP in human subjects.

(Angiotensinogen, Homo sapiens; GenBank:
AAA51679.1)
SEQ ID NO: 19
MRKRAPQSEMAPAGVSLRATILCLLAWAGLAAGDRVYIHPFHLVIHNEST
CEQLAKANAGKPKDPIFIPAPIQAKTSPVDEKALQDQLVLVAAKLDTEDK
LRAAMVGMLANFLGFRIYGMHSELWGVVHGATVLSPTAVFGTLASLYLGA
LDHTADRLQAILGVPWKDKNCTSRLDAHKVLSALQAVQGLLVAQGRADSQ
AQLLLSTVVGVFTAPGLHLKQPFVQGLALYTPVVLPRSLDFTELDVAAEK
IDRFMQAVTGWKTGCSLMGASVDSTLAFNTYVHFQGKMKGFSLLAEPQEF
WVDNSTSVSVPMLSGMGTFQHWSDIQDNFSVTEVPFTESACLLLIQPHYA
SDLDKVEGLTFQQNSLNWMKKLSPRTIHLTMPQLVLQGSYDLQDLLAQAE
LPAILHTELNLQKLSNDRIRVGEVLNSIFFELEADEREPTESTQQLNKPE
VLEVTLNRPFLFAVYDQSATALHFLGRVANPLSTA

The composition may be formulated with varying concentrations of the angiotensin therapeutic agent. In certain embodiments, the composition comprises angiotensin I or angiotensin II at a concentration of about 1, 2, 3, 4, 5, 6, 7, 8, 9, 10, 15, 20, 25, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, 1000 μg/ml. In certain embodiments, the composition comprises angiotensin III at a concentration of about 1, 2, 3, 4, 5, 6, 7, 8, 9, 10, 15, 20, 25, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, 1000, 1100, 1200, 1300, 1400, 1500, 1600, 1700, 1800, 1900, or 2000 μg/ml. In some embodiments, the composition comprises angiotensin IV or angiotensinogen at a concentration of about 1, 2, 3, 4, 5, 6, 7, 8, 9, 10, 15, 20, 25, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, or 4000 μg/ml. In other embodiments, the composition comprises the angiotensin therapeutic agent (e.g., angiotensin II) at a concentration of about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, or 25.0 mg/ml. In certain preferred embodiments, the composition comprises angiotensin II at a concentration of about 2.5 mg/mL.

In certain embodiments, the composition comprises an excipient, such as mannitol.

In certain embodiments, the composition is suitable for parenteral administration, such as injection or intravenous infusion, preferably intravenous infusion.

In some embodiments, the patient has sepsis. The patient may have septic shock, distributive shock, or cardiogenic shock.

In some embodiments, the patient is a mammal, such as a primate, ovine, porcine, canine, or rodent, preferably a human.

In some embodiments, the patient has acute respiratory distress syndrome (ARDS). In other embodiments, the patient does not have ARDS.

In some embodiments, the patient received an angiotensin converting enzyme (ACE) inhibitor 120, 72, 48, 24, 12, 10, 8, 6, or 4 hours prior to receiving the composition. In other embodiments, the patient has not received an ACE inhibitor 120, 72, 48, 24, 12, 10, 8, 6, or 4 hours prior to receiving the composition.

The rate of administration of the angiotensin therapeutic agent can be modulated manually and/or automatically in response to measurements of the mean arterial pressure of a patient obtained periodically or sporadically during treatment, e.g., to maintain a mean arterial pressure at this level, or within a predetermined range (e.g., 80-110 mm Hg).

In certain embodiments, the invention provides a method of assessing the response of a patient (such as a human) with hypotension to therapy with an angiotensin therapeutic agent, comprising administering to the patient an initial dose of a composition comprising an angiotensin therapeutic agent (which may be a therapeutic dose or a sub-therapeutic dose, for example, a dose less than 1 ng/kg/min or about 1 ng/kg/min) and testing the patient for a change in a therapeutic parameter (e.g., blood pressure). For example, the therapeutic parameter of the patient can be assessed prior to administering the initial dose and again after administering the initial dose (e.g., at least half an hour later, preferably at least one hour later and/or up to 8 hours later, preferably up to 6 hours later, such as between 1 and 6 hours after administering the initial dose). Comparing the assessment of the therapeutic parameter after administering the initial dose to the assessment made prior to administering the initial dose will indicate whether the parameter is increasing or decreasing as a result of the therapy with the angiotensin therapeutic agent. Typically, an increase in the blood pressure of a patient is indicative of a positive response to therapy with an angiotensin therapeutic agent. In certain embodiments, where the patient exhibits a positive response to the therapy, the method further comprises administering an additional dose of an angiotensin therapeutic agent to the patient. If a patient exhibits a negative response (e.g., a decrease in the patient's blood pressure), the patient will typically receive no additional doses of the angiotensin therapeutic agent. If a patient exhibits no response or an insignificant response, the method may further comprise administering a higher dose of the composition than the initial dose and further testing the patient for a response to the higher dose. Alternatively, if the patient exhibits no response or an insignificant response, the patient may receive no further doses of an angiotensin therapeutic agent.

In some embodiments, the method further comprises measuring a feature in a patient prior to administering the composition comprising the angiotensin therapeutic agent. The feature may be, for example, a blood concentration of angiotensin II, a blood concentration of angiotensin I, or a ratio of blood concentration of angiotensin I to blood concentration of angiotensin II. The feature may be blood plasma renin activity, a blood concentration of angiotensin converting enzyme (ACE), a blood concentration of aldosterone, a blood concentration of anti-diuretic hormone (ADH), or a blood concentration of angiotensinogen. The method may comprise administering the angiotensin therapeutic agent at a first initial rate if the measurement is greater than a predetermined threshold level, and the method may comprise administering the angiotensin therapeutic agent at a second initial rate if the measurement is less than or equal to the predetermined threshold level. The initial rate is preferably higher (e.g., at least 15-40 ng/kg/min angiotensin I or angiotensin II; or at least 20-40 ng/kg/min angiotensin III or angiotensin IV) if the measurement suggests that the patient has no defect in his or her endogenous renin-angiotensin system (e.g., the blood concentration of angiotensin II is above a predetermined threshold level, the blood concentration of angiotensin I is below a predetermined threshold level, the ratio of blood concentration of angiotensin I to blood concentration of angiotensin II is below a predetermined threshold level, the blood concentration of angiotensin converting enzyme is above a predetermined threshold level, the blood concentration of aldosterone is above a predetermined threshold level, the blood concentration of anti-diuretic hormone is above a predetermined threshold level, or the blood concentration of angiotensinogen is below a predetermined threshold level). The initial rate is preferable lower (e.g., less than 15-20 ng/kg/min angiotensin II; or less than 20-40 ng/kg/min angiotensin III or angiotensin IV) if the measurement suggests that the patient has a defect in his or her endogenous renin-angiotensin system (e.g., the blood concentration of angiotensin II is below a predetermined threshold level, the blood concentration of angiotensin I is above a predetermined threshold level, the ratio of blood concentration of angiotensin I to blood concentration of angiotensin II is above a predetermined threshold level, the blood concentration of angiotensin converting enzyme is below a predetermined threshold level, the blood concentration of aldosterone is below a predetermined threshold level, the blood concentration of anti-diuretic hormone is below a predetermined threshold level, or the blood concentration of angiotensinogen is above a predetermined threshold level).

Measuring a feature may comprise measuring a blood concentration of angiotensin II. The method may comprise administering the angiotensin therapeutic agent (e.g., angiotensin I or angiotensin II) at an initial rate (e.g., less than 1, 2, 3, 4, 5, 6, 7, 8, 9, 10, 11, 12, 13, 14, 15, 16, 17, 18, 19, 20, 21, 22, 23, 24, or 25 ng/kg/min), such as less than 20 ng/kg/min, if the blood concentration of angiotensin II is less than or equal to a predetermined threshold value (e.g., 1, 5, 10, 15, 20, 25, 30, 35, 40, 45, 50, 55, 60, 65, 70, 75, 80, 85, 90, 95, 100, 200, 500, or 1000 ng/mL), such as 50 ng/mL. The method may comprise administering the angiotensin therapeutic agent (e.g., angiotensinogen, angiotensin III, or angiotensin IV) at an initial rate (e.g., less than 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/kg/min), such as less than 40 ng/kg/min, if the blood concentration of angiotensin II is less than or equal to a predetermined threshold value (e.g., 1, 5, 10, 15, 20, 25, 30, 35, 40, 45, 50, 55, 60, 65, 70, 75, 80, 85, 90, 95, 100, 200, 500, or 1000 ng/mL), such as 50 ng/mL. The method may comprise administering the angiotensin therapeutic agent (e.g., angiotensin I or angiotensin II) at an initial rate (e.g., at least 1, 2, 3, 4, 5, 6, 7, 8, 9, 10, 11, 12, 13, 14, 15, 16, 17, 18, 19, 20, 21, 22, 23, 24, or 25 ng/kg/min), such as at least 20 ng/kg/min, if the blood concentration of angiotensin II is greater than a predetermined threshold value (e.g., 1, 5, 10, 15, 20, 25, 30, 35, 40, 45, 50, 55, 60, 65, 70, 75, 80, 85, 90, 95, 100, 200, 500, or 1000 ng/mL), such as 50 ng/mL. The method may comprise administering the angiotensin therapeutic agent (e.g., angiotensinogen, angiotensin III, or angiotensin IV) at an initial rate (e.g., at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/kg/min), such as at least 40 ng/kg/min, if the blood concentration of angiotensin II is greater than a predetermined threshold value (e.g., 1, 5, 10, 15, 20, 25, 30, 35, 40, 45, 50, 55, 60, 65, 70, 75, 80, 85, 90, 95, 100, 200, 500, or 1000 ng/mL), such as 50 ng/mL.

Measuring a feature may comprise measuring a blood concentration of angiotensin I. The method may comprise administering the angiotensin therapeutic agent (e.g., angiotensin II) at an initial rate (e.g., less than 1, 2, 3, 4, 5, 6, 7, 8, 9, 10, 11, 12, 13, 14, 15, 16, 17, 18, 19, 20, 21, 22, 23, 24, or 25 ng/kg/min), such as less than 20 ng/kg/min, if the blood concentration of angiotensin I is at least a predetermined threshold value (e.g., 10, 50, 100, 150, 200, 250, 300, 350, 400, 450, 500, 550, 600, 650, 700, 750, 800, 850, 900, 950, 1000, or 5000 pg/mL), such as 500 pg/mL. The method may comprise administering the angiotensin therapeutic agent (e.g., angiotensin III or angiotensin IV) at an initial rate (e.g., less than 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/kg/min), such as less than 40 ng/kg/min, if the blood concentration of angiotensin I is at least a predetermined threshold value (e.g., 10, 50, 100, 150, 200, 250, 300, 350, 400, 450, 500, 550, 600, 650, 700, 750, 800, 850, 900, 950, 1000, or 5000 pg/mL), such as 500 pg/mL. The method may comprise administering the angiotensin therapeutic agent (e.g., angiotensin I or angiotensin II) at an initial rate (e.g., at least 1, 2, 3, 4, 5, 6, 7, 8, 9, 10, 11, 12, 13, 14, 15, 16, 17, 18, 19, 20, 21, 22, 23, 24, or 25 ng/kg/min), such as at least 20 ng/kg/min, if the blood concentration of angiotensin I is less than a predetermined threshold value (e.g., 10, 50, 100, 150, 200, 250, 300, 350, 400, 450, 500, 550, 600, 650, 700, 750, 800, 850, 900, 950, 1000, or 5000 pg/mL), such as 500 pg/mL. The method may comprise administering the angiotensin therapeutic agent (e.g., angiotensinogen, angiotensin III, or angiotensin IV) at an initial rate (e.g., at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/kg/min), such as at least 40 ng/kg/min, if the blood concentration of angiotensin I is less than a predetermined threshold value (e.g., 10, 50, 100, 150, 200, 250, 300, 350, 400, 450, 500, 550, 600, 650, 700, 750, 800, 850, 900, 950, 1000, or 5000 pg/mL), such as 500 pg/mL.

Measuring a feature may comprise measuring a ratio of angiotensin I to angiotensin II. The method may comprise administering the angiotensin therapeutic agent (e.g., angiotensin II) at an initial rate (e.g., less than 1, 2, 3, 4, 5, 6, 7, 8, 9, 10, 11, 12, 13, 14, 15, 16, 17, 18, 19, 20, 21, 22, 23, 24, or 25 ng/kg/min), such as less than 20 ng/kg/min, if the ratio of angiotensin I to angiotensin II is at least a predetermined threshold value (e.g., 1:100, 1:50, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 50:1, or 100:1), such as 1:1. The method may comprise administering the angiotensin therapeutic agent (e.g., angiotensin III or angiotensin IV) at an initial rate (e.g., less than 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/kg/min), such as less than 40 ng/kg/min, if the ratio of angiotensin I to angiotensin II is at least a predetermined threshold value (e.g., 1:100, 1:50, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 50:1, or 100:1), such as 1:1. The method may comprise administering the angiotensin therapeutic agent (e.g., angiotensin I or angiotensin II) at an initial rate (e.g., at least 1, 2, 3, 4, 5, 6, 7, 8, 9, 10, 11, 12, 13, 14, 15, 16, 17, 18, 19, 20, 21, 22, 23, 24, or 25 ng/kg/min), such as at least 20 ng/kg/min, if the ratio of angiotensin I to angiotensin II is less than a predetermined threshold value (e.g., 1:100, 1:50, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 50:1, or 100:1), such as 1:1. The method may comprise administering the angiotensin therapeutic agent (e.g., angiotensinogen, angiotensin III, or angiotensin IV) at an initial rate (e.g., at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/kg/min), such as at least 40 ng/kg/min, if the ratio of angiotensin I to angiotensin II is less than a predetermined threshold value (e.g., 1:100, 1:50, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 50:1, or 100:1), such as 1:1.

Measuring a feature may comprise measuring a blood plasma renin activity. The method may comprise administering the angiotensin therapeutic agent (e.g., angiotensin I or angiotensin II) at an initial rate (e.g., less than 1, 2, 3, 4, 5, 6, 7, 8, 9, 10, 11, 12, 13, 14, 15, 16, 17, 18, 19, 20, 21, 22, 23, 24, or 25 ng/kg/min), such as less than 20 ng/kg/min, if the blood plasma renin activity is less than a predetermined threshold value (e.g., 50, 20, 10, 5, 4, 3, 2, 1.5, 1.2, 1.1, 1.0, 0.9, 0.8, 0.7, 0.6, 0.5, 0.4, 0.3, 0.2, 0.1, 0.05, or 0.01 μIU/mL), such as 1.2 μIU/mL. The method may comprise administering the angiotensin therapeutic agent (e.g., angiotensin III, or angiotensin IV) at an initial rate (e.g., less than 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/kg/min), such as less than 40 ng/kg/min, if the blood plasma renin activity is less than a predetermined threshold value (e.g., 50, 20, 10, 5, 4, 3, 2, 1.5, 1.2, 1.1, 1.0, 0.9, 0.8, 0.7, 0.6, 0.5, 0.4, 0.3, 0.2, 0.1, 0.05, or 0.01 μIU/mL), such as 1.2 μIU/mL. The method may comprise administering the angiotensin therapeutic agent (e.g., angiotensin I or angiotensin II) at an initial rate (e.g., at least 1, 2, 3, 4, 5, 6, 7, 8, 9, 10, 11, 12, 13, 14, 15, 16, 17, 18, 19, 20, 21, 22, 23, 24, or 25 ng/kg/min), such as at least 20 ng/kg/min, if the blood plasma renin activity is at least a predetermined threshold value (e.g., 50, 20, 10, 5, 4, 3, 2, 1.5, 1.2, 1.1, 1.0, 0.9, 0.8, 0.7, 0.6, 0.5, 0.4, 0.3, 0.2, 0.1, 0.05, or 0.01 μIU/mL), such as 1.2 μIU/mL. The method may comprise administering the angiotensin therapeutic agent (e.g., angiotensinogen, angiotensin III, or angiotensin IV) at an initial rate (e.g., at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/kg/min), such as at least 40 ng/kg/min, if the blood plasma renin activity is at least a predetermined threshold value (e.g., 50, 20, 10, 5, 4, 3, 2, 1.5, 1.2, 1.1, 1.0, 0.9, 0.8, 0.7, 0.6, 0.5, 0.4, 0.3, 0.2, 0.1, 0.05, or 0.01 μIU/mL), such as 1.2 μIU/mL.

Measuring a feature may comprise measuring the blood concentration of angiotensin converting enzyme (ACE). The method may comprise administering the angiotensin therapeutic agent (e.g., angiotensin II) at an initial rate (e.g., less than 1, 2, 3, 4, 5, 6, 7, 8, 9, 10, 11, 12, 13, 14, 15, 16, 17, 18, 19, 20, 21, 22, 23, 24, or 25 ng/kg/min), such as less than 20 ng/kg/min, if the blood concentration of ACE is less than a predetermined threshold value (e.g., 0.5, 1, 5, 10, 15, 20, 25, 30, 35, 40, 45, 50, 55, 60, 65, 70, 75, 80, 85, 90, 95, 100, 200, 500, or 1000 nmol/mL), such as 40 nmol/mL. The method may comprise administering the angiotensin therapeutic agent (e.g., angiotensin III or angiotensin IV) at an initial rate (e.g., less than 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/kg/min), such as less than 40 ng/kg/min, if the blood concentration of ACE is less than a predetermined threshold value (e.g., 0.5, 1, 5, 10, 15, 20, 25, 30, 35, 40, 45, 50, 55, 60, 65, 70, 75, 80, 85, 90, 95, 100, 200, 500, or 1000 nmol/mL), such as 40 nmol/mL. The method may comprise administering the angiotensin therapeutic agent (e.g., angiotensin I or angiotensin II) at an initial rate (e.g., at least 1, 2, 3, 4, 5, 6, 7, 8, 9, 10, 11, 12, 13, 14, 15, 16, 17, 18, 19, 20, 21, 22, 23, 24, or 25 ng/kg/min), such as at least 20 ng/kg/min, if the blood concentration of ACE is at least a predetermined threshold value (e.g., 0.5, 1, 5, 10, 15, 20, 25, 30, 35, 40, 45, 50, 55, 60, 65, 70, 75, 80, 85, 90, 95, 100, 200, 500, or 1000 nmol/mL), such as 40 nmol/mL. The method may comprise administering the angiotensin therapeutic agent (e.g., angiotensinogen, angiotensin III, or angiotensin IV) at an initial rate (e.g., at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/kg/min), such as at least 40 ng/kg/min, if the blood concentration of ACE is at least a predetermined threshold value (e.g., 0.5, 1, 5, 10, 15, 20, 25, 30, 35, 40, 45, 50, 55, 60, 65, 70, 75, 80, 85, 90, 95, 100, 200, 500, or 1000 nmol/mL), such as 40 nmol/mL.

Measuring a feature may comprise measuring the blood concentration of aldosterone. The method may comprise administering the angiotensin therapeutic agent (e.g., angiotensin I or angiotensin II) at an initial rate (e.g., less than 1, 2, 3, 4, 5, 6, 7, 8, 9, 10, 11, 12, 13, 14, 15, 16, 17, 18, 19, 20, 21, 22, 23, 24, or 25 ng/kg/min), such as less than 20 ng/kg/min, if the blood concentration of aldosterone is less than a predetermined threshold value (e.g., 0.1, 0.5, 1, 2, 3, 4, 5, 6, 7, 8, 9, 10, 11, 12, 13, 14, 15, 16, 17, 18, 19, 20, 40, 50, 80, or 100 ng/dL), such as 5 ng/dL. The method may comprise administering the angiotensin therapeutic agent (e.g., angiotensinogen, angiotensin III, or angiotensin IV) at an initial rate (e.g., less than 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/kg/min), such as less than 40 ng/kg/min, if the blood concentration of aldosterone is less than a predetermined threshold value (e.g., 0.1, 0.5, 1, 2, 3, 4, 5, 6, 7, 8, 9, 10, 11, 12, 13, 14, 15, 16, 17, 18, 19, 20, 40, 50, 80, or 100 ng/dL), such as 5 ng/dL. The method may comprise administering the angiotensin therapeutic agent (e.g., angiotensin I or angiotensin II) at an initial rate (e.g., at least 1, 2, 3, 4, 5, 6, 7, 8, 9, 10, 11, 12, 13, 14, 15, 16, 17, 18, 19, 20, 21, 22, 23, 24, or 25 ng/kg/min), such as at least 20 ng/kg/min, if the blood concentration of aldosterone is at least a predetermined threshold value (e.g., 0.1, 0.5, 1, 2, 3, 4, 5, 6, 7, 8, 9, 10, 11, 12, 13, 14, 15, 16, 17, 18, 19, 20, 40, 50, 80, or 100 ng/dL), such as 5 ng/dL. The method may comprise administering the angiotensin therapeutic agent (e.g., angiotensinogen, angiotensin III, or angiotensin IV) at an initial rate (e.g., at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/kg/min), such as at least 40 ng/kg/min, if the blood concentration of aldosterone is at least a predetermined threshold value (e.g., 0.1, 0.5, 1, 2, 3, 4, 5, 6, 7, 8, 9, 10, 11, 12, 13, 14, 15, 16, 17, 18, 19, 20, 40, 50, 80, or 100 ng/dL), such as 5 ng/dL.

Measuring a feature may comprise measuring the blood concentration of anti-diuretic hormone (ADH). The method may comprise administering the angiotensin therapeutic agent (e.g., angiotensin I or angiotensin II) at an initial rate (e.g., less than 1, 2, 3, 4, 5, 6, 7, 8, 9, 10, 11, 12, 13, 14, 15, 16, 17, 18, 19, 20, 21, 22, 23, 24, or 25 ng/kg/min), such as less than 20 ng/kg/min, if the blood concentration of ADH is less than a predetermined threshold value (e.g., 0.05, 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11, 12, 13, 14, 15, 20, 25, 30, 40, 50, or 100 pg/mL), such as 2.5 pg/mL. The method may comprise administering the angiotensin therapeutic agent (e.g., angiotensinogen, angiotensin III, or angiotensin IV) at an initial rate (e.g., less than 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/kg/min), such as less than 40 ng/kg/min, if the blood concentration of ADH is less than a predetermined threshold value (e.g., 0.05, 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11, 12, 13, 14, 15, 20, 25, 30, 40, 50, or 100 pg/mL), such as 2.5 pg/mL. The method may comprise administering the angiotensin therapeutic agent (e.g., angiotensin I or angiotensin II) at an initial rate (e.g., at least 1, 2, 3, 4, 5, 6, 7, 8, 9, 10, 11, 12, 13, 14, 15, 16, 17, 18, 19, 20, 21, 22, 23, 24, or 25 ng/kg/min), such as at least 20 ng/kg/min, if the blood concentration of ADH is at least a predetermined threshold value (e.g., 0.05, 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11, 12, 13, 14, 15, 20, 25, 30, 40, 50, or 100 pg/mL), such as 2.5 pg/mL. The method may comprise administering the angiotensin therapeutic agent (e.g., angiotensinogen, angiotensin III, or angiotensin IV) at an initial rate (e.g., at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/kg/min), such as at least 40 ng/kg/min, if the blood concentration of ADH is at least a predetermined threshold value (e.g., 0.05, 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11, 12, 13, 14, 15, 20, 25, 30, 40, 50, or 100 pg/mL), such as 2.5 pg/mL.

Measuring a feature may comprise measuring the blood concentration of angiotensinogen. The method may comprise administering the angiotensin therapeutic agent (e.g., angiotensin I or angiotensin II) at an initial rate (e.g., less than 1, 2, 3, 4, 5, 6, 7, 8, 9, 10, 11, 12, 13, 14, 15, 16, 17, 18, 19, 20, 21, 22, 23, 24, or 25 ng/kg/min), such as less than 20 ng/kg/min, if the blood concentration of angiotensinogen is at least a predetermined threshold value (e.g., 5, 10, 50, 100, 150, 200, 250, 300, 350, 400, 450, 500, 550, 600, 650, 700, 750, 800, 850, 900, 950, 1000, 2000, 5000, or 10,000 ng/mL), such as 250 ng/mL. The method may comprise administering the angiotensin therapeutic agent (e.g., angiotensin III or angiotensin IV) at an initial rate (e.g., less than 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/kg/min), such as less than 40 ng/kg/min, if the blood concentration of angiotensinogen is at least a predetermined threshold value (e.g., 5, 10, 50, 100, 150, 200, 250, 300, 350, 400, 450, 500, 550, 600, 650, 700, 750, 800, 850, 900, 950, 1000, 2000, 5000, or 10,000 ng/mL), such as 250 ng/mL. The method may comprise administering the angiotensin therapeutic agent (e.g., angiotensin I or angiotensin II) at an initial rate (e.g., at least 1, 2, 3, 4, 5, 6, 7, 8, 9, 10, 11, 12, 13, 14, 15, 16, 17, 18, 19, 20, 21, 22, 23, 24, or 25 ng/kg/min), such as at least 20 ng/kg/min, if the blood concentration of angiotensinogen is less than a predetermined threshold value (e.g., 5, 10, 50, 100, 150, 200, 250, 300, 350, 400, 450, 500, 550, 600, 650, 700, 750, 800, 850, 900, 950, 1000, 2000, 5000, or 10,000 ng/mL), such as 250 ng/mL. The method may comprise administering the angiotensin therapeutic agent (e.g., angiotensinogen, angiotensin III, or angiotensin IV) at an initial rate (e.g., at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/kg/min), such as at least 40 ng/kg/min, if the blood concentration of angiotensinogen is less than a predetermined threshold value (e.g., 5, 10, 50, 100, 150, 200, 250, 300, 350, 400, 450, 500, 550, 600, 650, 700, 750, 800, 850, 900, 950, 1000, 2000, 5000, or 10,000 ng/mL), such as 250 ng/mL.

The method may further comprise assessing the lung function of the patient, wherein assessing lung function comprises determining whether a patient has impaired lung function. For example, a patient may have impaired lung function if the patient has an acute or chronic lung condition selected from a respiratory disease, an inflammatory lung disease, a respiratory tract infection, a restrictive lung disease, lung cancer, a pleural cavity disease, a pulmonary vascular disease (such as a pulmonary embolism), acute respiratory distress syndrome, or lung trauma. The initial rate is preferably higher if the patient does not have impaired lung function (e.g., at least 15-40 ng/kg/min angiotensin I or angiotensin II; or at least 20-40 ng/kg/min angiotensin III or angiotensin IV). The initial rate is preferable lower if the patient has impaired lung function (e.g., less than 15-20 ng/kg/min angiotensin II; or less than 20-40 ng/kg/min angiotensin III or angiotensin IV).

The method may comprise administering the angiotensin therapeutic agent (e.g., angiotensin II) at an initial rate (e.g., less than 1, 2, 3, 4, 5, 6, 7, 8, 9, 10, 11, 12, 13, 14, 15, 16, 17, 18, 19, 20, 21, 22, 23, 24, or 25 ng/kg/min), such as less than 20 ng/kg/min, if the patient has impaired lung function. The method may comprise administering the angiotensin therapeutic agent (e.g., angiotensin III or angiotensin IV) at an initial rate (e.g., less than 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/kg/min), such as less than 40 ng/kg/min, if the patient has impaired lung function. The method may comprise administering the angiotensin therapeutic agent (e.g., angiotensin I or angiotensin II) at an initial rate (e.g., at least 1, 2, 3, 4, 5, 6, 7, 8, 9, 10, 11, 12, 13, 14, 15, 16, 17, 18, 19, 20, 21, 22, 23, 24, or 25 ng/kg/min), such as at least 20 ng/kg/min, if the patient does not have impaired lung function. The method may comprise administering the angiotensin therapeutic agent (e.g., angiotensinogen, angiotensin III, or angiotensin IV) at an initial rate (e.g., at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/kg/min), such as at least 40 ng/kg/min, if the patient does not have impaired lung function.

The method may further comprise determining whether a patient has acute respiratory distress syndrome. The initial rate is preferably higher if the patient does not have acute respiratory distress syndrome (e.g., at least 15-40 ng/kg/min angiotensin I or angiotensin II; or at least 20-40 ng/kg/min angiotensin III or angiotensin IV). The initial rate is preferable lower if the patient has acute respiratory distress syndrome (e.g., less than 15-20 ng/kg/min angiotensin II; or less than 20-40 ng/kg/min angiotensin III or angiotensin IV). The method may comprise administering the angiotensin therapeutic agent (e.g., angiotensin II) at an initial rate (e.g., less than 1, 2, 3, 4, 5, 6, 7, 8, 9, 10, 11, 12, 13, 14, 15, 16, 17, 18, 19, 20, 21, 22, 23, 24, or 25 ng/kg/min), such as less than 20 ng/kg/min, if the patient has acute respiratory distress syndrome. The method may comprise administering the angiotensin therapeutic agent (e.g., angiotensin III or angiotensin IV) at an initial rate (e.g., less than 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/kg/min), such as less than 40 ng/kg/min, if the patient has acute respiratory distress syndrome. The method may comprise administering the angiotensin therapeutic agent (e.g., angiotensin I or angiotensin II) at an initial rate (e.g., at least 1, 2, 3, 4, 5, 6, 7, 8, 9, 10, 11, 12, 13, 14, 15, 16, 17, 18, 19, 20, 21, 22, 23, 24, or 25 ng/kg/min), such as at least 20 ng/kg/min, if the patient does not have acute respiratory distress syndrome. The method may comprise administering the angiotensin therapeutic agent (e.g., angiotensinogen, angiotensin III, or angiotensin IV) at an initial rate (e.g., at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/kg/min), such as at least 40 ng/kg/min, if the patient does not have acute respiratory distress syndrome.

The method may further comprise determining whether the patient received an angiotensin converting enzyme inhibitor (ACE inhibitor) within a preceding period of time. The initial rate is preferably higher if the patient has not received an angiotensin converting enzyme inhibitor within the preceding period of time (e.g., at least 15-40 ng/kg/min angiotensin I or angiotensin II; or at least 20-40 ng/kg/min angiotensin III or angiotensin IV). The initial rate is preferable lower if the patient received an angiotensin converting enzyme inhibitor within the preceding period of time (e.g., less than 15-20 ng/kg/min angiotensin II; or less than 20-40 ng/kg/min angiotensin III or angiotensin IV). The preceding period of time may be about 1 hour to about 72 hours, such as about 1 hour to about 48 hours, about 1 hour to about 24 hours, about 1 hour to about 12 hours, or about 1 hour to about 6 hours. The preceding period of time may be less than 72 hours, less than 48 hours, less than 24 hours, less than 12 hours, or less than 6 hours. The ACE inhibitor may be selected from perindopril, captopril, enalapril, lisinopril, benazepril, fosinopril, moexipril, quinapril, trandolapril, and ramipril.

The method may comprise administering the angiotensin therapeutic agent (e.g., angiotensin II) at an initial rate (e.g., less than 1, 2, 3, 4, 5, 6, 7, 8, 9, 10, 11, 12, 13, 14, 15, 16, 17, 18, 19, 20, 21, 22, 23, 24, or 25 ng/kg/min), such as less than 20 ng/kg/min, if the patient received an ACE inhibitor within the preceding period of time. The method may comprise administering the angiotensin therapeutic agent (e.g., angiotensin III or angiotensin IV) at an initial rate (e.g., less than 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/kg/min), such as less than 40 ng/kg/min, if the patient received an ACE inhibitor within the preceding period of time. The method may comprise administering the angiotensin therapeutic agent (e.g., angiotensin I or angiotensin II) at an initial rate (e.g., at least 1, 2, 3, 4, 5, 6, 7, 8, 9, 10, 11, 12, 13, 14, 15, 16, 17, 18, 19, 20, 21, 22, 23, 24, or 25 ng/kg/min), such as at least 20 ng/kg/min, if the patient did not receive an ACE inhibitor within the preceding period of time. The method may comprise administering the angiotensin therapeutic agent (e.g., angiotensinogen, angiotensin III, or angiotensin IV) at an initial rate (e.g., at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/kg/min), such as at least 40 ng/kg/min, if the patient did not receive an ACE inhibitor within the preceding period of time.

### Angiotensin Therapeutics

Angiotensinogen, angiotensin I, angiotensin II, angiotensin III, and angiotensin IV are hormones naturally produced by the body that regulate blood pressure via vasoconstriction and sodium reabsorption. Hemodynamic effects of angiotensin II administration have been the patient of numerous clinical studies, demonstrating significant effects on systemic and renal blood flow (Harrison-Bernard, L. M., The renal renin-angiotensin system. Adv Physiol Educ, (2009) 33(4):270-74). The angiotensins are hormones produced by the renin angiotensin aldosterone system (RAAS) that modulates blood pressure via regulation of vascular smooth muscle tone and extracellular fluid homeostasis. The term “angiotensin therapeutic agent,” without further specificity, in this disclosure refers to angiotensinogen, angiotensin I, angiotensin II, angiotensin III, and/or angiotensin IV and analogs and mixtures thereof. Angiotensin therapeutic agents mediate their effects on the vasculature by inducing vasoconstriction and sodium retention, and so is the target of many therapies for hypertension. In addition to its systemic effects, angiotensin therapeutic agents display a pronounced effect on the efferent arterioles of the kidney, maintaining glomerular filtration when blood flow is decreased. Angiotensin II also regulates sodium reabsorption in the kidney by stimulating Na⁺/H⁺ exchangers in the proximal tubule and inducing the release of aldosterone and vasopressin (Harrison-Bernard, L. M., The renal renin-angiotensin system. Adv Physiol Educ, (2009) 33(4):270-4.).

The angiotensin therapeutic agent that may be used in the compositions and methods of this disclosure may be Asp-Arg-Val-Tyr-Ile-His-Pro-Phe (SEQ ID NO: 1) also called 5-isoleucine angiotensin II, 5-L-isoleucine angiotensin II, 1-aspartate-5-isoleucine angiotensin II, and 1-L-aspartate-5-L-isoleucine angiotensin II. SEQ ID NO:1 is an octa-peptide naturally present in humans and other species, such as equines, hogs, etc. Isoleucine may be substituted by valine to result in 5-L-valine angiotensin II, Asp-Arg-Val-Tyr-Val-His-Pro-Phe (SEQ ID NO:2). Other angiotensin II analogs such as [Asn¹-Phe⁴]-angiotensin II (SEQ ID NO:3), nonapeptide Asn-Arg-Val-Tyr-Tyr-Val-His-Pro-Phe (SEQ ID NO:4), [Asn¹-Ile⁵-Ile⁸]-angiotensin II (SEQ ID NO:5), [Asn¹-Ile⁵-Ala⁸]-angiotensin II (SEQ ID NO:6), and [Asn¹-diiodoTyr⁴-Ile⁵]-angiotensin II (SEQ ID NO:7) may also be used. Angiotensin II may be synthesized, for example, by solid phase peptide synthesis to incorporate modifications, such as C-terminal amidation, N-terminal acetylation, or diiodo-tyrosine. The term “angiotensin II,” without further specificity, is intended to refer to any of these various forms, as well as combinations thereof.

In some aspects, the angiotensin therapeutic agent may be selected from 5-L-valine angiotensin II, 5-L-valine angiotensin II amide, 5-L-isoleucine angiotensin II, and 5-L-isoleucine angiotensin II amide, or a pharmaceutically acceptable salt thereof, preferably manufactured under current good manufacturing conditions (cGMP). In some aspects, the composition may include different forms of angiotensin II in different percentages, e.g., a mixture 5-L-valine angiotensin II and 5-L-isoleucine angiotensin II. In some embodiments, the composition includes a mixture of angiotensinogen, angiotensin I, angiotensin II, angiotensin, III, and/or angiotensin IV. For example, the composition may include a mixture of different forms of angiotensinogen, angiotensin I, angiotensin II, angiotensin III, and/or angiotensin IV at varying percentages. The composition comprising the angiotensin therapeutic agent may be suitable for parenteral administration, e.g., for injection or intravenous infusion.

An angiotensin III therapeutic that may be used in the compositions and methods of this disclosure may be Arg-Val-Tyr-Ile-His-Pro-Phe (SEQ ID NO:8). SEQ ID NO:8 is a hepta-peptide naturally present in humans and other species, such as equines, hogs, etc. Isoleucine may be substituted by valine to result in Arg-Val-Tyr-Val-His-Pro-Phe (SEQ ID NO:9). Other angiotensin III analogs such as [Phe³]-angiotensin III (SEQ ID NO:10), [Ile⁴-Ala⁷]-angiotensin III (SEQ ID NO:11), and [diiodoTyr³-Ile⁴]-angiotensin III (SEQ ID NO:12) may also be used. Angiotensin III may be synthesized, for example, by solid phase peptide synthesis to incorporate modifications, such as C-terminal amidation, N-terminal acetylation, or diiodo-tyrosine. The term “angiotensin III,” without further specificity, is intended to refer to any of these various forms, as well as combinations thereof.

In some aspects, the angiotensin therapeutic agent may be selected from 4-L-valine angiotensin III, 4-L-valine angiotensin III amide, 4-L-isoleucine angiotensin III, and 4-L-isoleucine angiotensin III amide, or a pharmaceutically acceptable salt thereof, preferably manufactured under current good manufacturing conditions (cGMP).

Angiotensin IV is a metabolite of angiotensin III. An angiotensin IV therapeutic that may be used in the compositions and methods of this disclosure may be Val-Tyr-Ile-His-Pro-Phe (SEQ ID NO:13). SEQ ID NO:13 is a hexa-peptide naturally present in humans and other species, such as equines, hogs, etc. Isoleucine may be substituted by valine to result in Val-Tyr-Val-His-Pro-Phe (SEQ ID NO:14). Other angiotensin IV analogs such as [Phe²]-angiotensin IV (SEQ ID NO:15), [Ile³-Ala⁶]-angiotensin IV (SEQ ID NO:16), and [diiodoTyr²-Ile³]-angiotensin IV (SEQ ID NO:17) may also be used. Angiotensin IV may be synthesized, for example, by solid phase peptide synthesis to incorporate modifications, such as C-terminal amidation, N-terminal acetylation, or diiodo-tyrosine. The term “angiotensin IV,” without further specificity, is intended to refer to any of these various forms, as well as combinations thereof.

In some aspects, a composition comprising angiotensin IV may be selected from 3-L-valine angiotensin IV, 3-L-valine angiotensin IV amide, 3-L-isoleucine angiotensin IV, and 3-L-isoleucine angiotensin IV amide, or a pharmaceutically acceptable salt thereof, preferably manufactured under current good manufacturing conditions (cGMP).

The angiotensin therapeutic agent that may be used in the compositions and methods of this disclosure may be Asp-Arg-Val-Tyr-Ile-His-Pro-Phe-His-Leu (SEQ ID NO:18) also called 5-L-isoleucine angiotensin I. SEQ ID NO:18 is an deca-peptide naturally present in humans and other species, such as equines, hogs, etc. Isoleucine may be substituted by valine to result in 5-L-valine angiotensin I, Asp-Arg-Val-Tyr-Val-His-Pro-Phe-His-Leu (SEQ ID NO:19). Other angiotensin I analogs such as [Asn¹-Phe⁴]-angiotensin I (SEQ ID NO:20), nona-peptide Arg-Val-Tyr-Ile-His-Pro-Phe-His-Leu (SEQ ID NO:21), octa-peptide Val-Tyr-Ile-His-Pro-Phe-His-Leu (SEQ ID NO:22), [Asn¹]-angiotensin I Asn-Arg-Val-Tyr-Tyr-Val-His-Pro-Phe-His-Leu (SEQ ID NO:23), [Asn¹-Ile⁵-Ile⁸]-angiotensin I (SEQ ID NO:24), [Asn¹-Ile⁵-Ala⁸]-angiotensin I (SEQ ID NO:25), and [Asn¹-diiodoTyr⁴-Ile⁵]-angiotensin I (SEQ ID NO:26) may also be used. Angiotensin I may be synthesized, for example, by solid phase peptide synthesis to incorporate modifications, such as C-terminal amidation, N-terminal acetylation, or diiodo-tyrosine. The term “angiotensin I,” without further specificity, is intended to refer to any of these various forms, as well as combinations thereof.

An angiotensin therapeutic may be used as any suitable salt, deprotected form, acetylated form, deacetylated form, and/or prodrug form of the above-mentioned peptides, including pegylated forms of the peptides or conjugates as disclosed in US Patent Publication 2011/0081371 (hereby incorporated by reference in its entirety). The term “prodrug” refers to any precursor compound which is able to generate or to release the above-mentioned peptide under physiological conditions. Such prodrugs may be larger peptides which are selectively cleaved in order to form the peptide of the invention. For example, in some aspects, the prodrug may be angiotensinogen, angiotensin I, or its homologues that may result in the production of angiotensin II by the action of certain endogenous or exogenous enzymes. In another example, the prodrug may be angiotensin II, angiotensin III, or homologs thereof that may result in angiotensin III and angiotensin IV, respectively. Further prodrugs include peptides with protected amino acids, e.g., having protecting groups at one or more carboxylic acid and/or amino groups. Suitable protecting groups for amino groups are the benzyloxycarbonyl, t-butyloxycarbonyl (BOC), fluorenylmethyloxycarbonyl (FMOC), formyl, and acetyl or acyl group. Suitable protecting groups for the carboxylic acid group are esters such as benzyl esters or t-butyl esters. The present invention also contemplates the use of angiotensinogen, angiotensin I, angiotensin II, angiotensin III, and/or angiotensin IV and/or precursor peptides having amino acid substitutions, deletions, additions, the substitutions and additions including the standard D and L amino acids and modified amino acids, such as, for example, amidated and acetylated amino acids, wherein the therapeutic activity of the base peptide sequence is maintained at a pharmacologically useful level.

In some embodiments, the angiotensin therapeutic agent is a peptide or protein, wherein the N-terminus of the peptide or protein consists of the amino acid sequence set forth in any one of SEQ ID NO:1-26. In preferred embodiments, the N-terminus of the peptide or protein consists of the amino acid sequence set forth in SEQ ID NO:18. In more preferred embodiments, the N-terminus of the peptide or protein consists of the amino acid sequence set forth in SEQ ID NO:28 (Asp-Arg-Val-Tyr-Ile-His-Pro-Phe-His-Leu-Val-Ile). In certain preferred embodiments, the peptide or protein has at least 95% sequence homology with the sequence set forth in SEQ ID NO:27. For example, the peptide or protein may have at least 95%, at least 96%, at least 97%, at least 98%, or at least 99% sequence homology with the sequence set forth in SEQ ID NO:27. The N-terminus of the peptide or protein may consist of the amino acid sequence set forth in any one of SEQ ID NO:1-26 or SEQ ID NO:28, and the peptide or protein may have at least 95%, 96%, 97%, 98%, or 99% sequence homology with the sequence set forth in SEQ ID NO:27. The peptide or protein may be longer or shorter than the sequence set forth in SEQ ID NO:27, such as about 10 amino acids to about 2000 amino acids in length, about 100 to about 2000, about 100 to about 1500, about 100 to about 1000, about 200 to about 2000, about 200 to about 1500, about 200 to about 1000, about 500 to about 2000, about 500 to about 1500, about 500 to about 1000, about 10 to about 1000 amino acids, about 10 to about 500 amino acids, about 10 to about 400 amino acids, about 10 to about 300 amino acids, about 10 to about 200 amino acids, about 10 to about 100 amino acids, about 10 to about 50 amino acids, about 20 to about 500 amino acids, about 20 to about 400 amino acids, about 20 to about 300 amino acids, about 20 to about 200 amino acids, about 20 to about 100 amino acids, about 20 to about 50 amino acids, about 25 to about 500 amino acids, about 25 to about 400 amino acids, about 25 to about 300 amino acids, about 25 to about 200 amino acids, about 25 to about 100 amino acids, or about 25 to about 50 amino acids in length. For example, the N-terminus of the peptide or protein may consist of the amino acid sequence set forth in any one of SEQ ID NO:1-26 or SEQ ID NO:28; the peptide or protein may have at least 95%, 96%, 97%, 98%, or 99% sequence homology with the sequence set forth in SEQ ID NO:27; and the peptide or protein may be about 10 amino acids to about 2000 amino acids in length. The peptide or protein may comprise an antibody Fc fragment (e.g., at its C-terminus), for example, to increase the half-life of the therapeutic angiotensin agent in vivo. Such fusion proteins would be expected to have less than 10% sequence homology with SEQ ID NO:27. The peptide or protein may comprise one or more modifications, such as glycosylation and/or pegylation, e.g., which may result in more favorable pharmacokinetics and/or pharmacodynamics in certain patients relative to angiotensinogen, angiotensin I, angiotensin II, angiotensin III, and/or angiotensin IV.

In some embodiments, the peptide or protein has about 1% to about 1000% of the activity as angiotensin II, such as about 2% to about 500% or about 5% to about 200%. In preferred embodiments, the peptide or protein has about 10% to about 1000% of the activity as angiotensin II, such as about 20% to about 500% or about 30% to about 300%. Activity refers to the ability of the angiotensin therapeutic agent to raise the blood pressure (e.g., MAP) of a patient. For example, an angiotensin therapeutic agent that requires an administration rate that is 3 times greater than for angiotensin II, by weight, to raise the blood pressure of a patient by the same amount has about 33% of the activity of angiotensin II. The molecular weight of angiotensinogen is approximately 60 times the molecular weight of angiotensin II, for example, and thus, the activity of angiotensinogen is approximately 2% that of angiotensin II. A fusion peptide consisting of angiotensin II and the Fc fragment of an antibody has a similar molecular weight as angiotensinogen, and such a fusion peptide would be expected to display an activity greater than about 2% for fusions that display favorable pharmacokinetics.

### Doses of the Therapeutically Effective Substance

In general, an angiotensin therapeutic agent increases blood pressure, and patients who are hypotensive may require larger doses to exhibit pressor responses similar to those observed in normal patients. The composition including the angiotensin therapeutic agent can be administered at a rate sufficient to achieve an increase in blood pressure of at least about 10-15 mm Hg, and optionally, the rate at which the angiotensin therapeutic agent is administered may be varied in response to changes in other physiological parameters such as renal vascular resistance, renal blood flow, filtration fractions, mean arterial pressure, etc. For example, the rate of administration of the angiotensin therapeutic agent may start from about 2 ng/kg/min to about 20 ng/kg/min and is increased based on the mean arterial pressure (“MAP”). In some aspects, the rate of administration may be increased such that the MAP does not exceed about 70 mm Hg, about 80 mm Hg, about 90 mm Hg, about 100 mm Hg, about 110 mm Hg, etc. For example, a patient may be coupled to a monitor that provides continuous, periodic, or occasional measurements of MAP during some or all of the course of treatment. The rate of administration may be modulated manually (e.g., by a physician or nurse) or automatically (e.g., by a medical device capable of modulating delivery of the composition in response to MAP values received from the monitor) to maintain the MAP of a patient within a desired range (e.g., 80-110 mm Hg) or below a desired threshold, e.g., as set forth above.

The composition including the angiotensin therapeutic agent may be administered over a period of time selected from at least 8 hours; at least 24 hours; and from 8 hours to 24 hours. The composition including the angiotensin therapeutic agent may be administered continuously for at least 2-6 days, such as 2-11 days, continuously for 2-6 days, for 8 hours a day over a period of at least 2-6 days, such as 2-11 days. A weaning period (from several hours to several days) may be beneficial after prolonged infusion.

The composition including the angiotensin therapeutic agent may further include one or more additional pharmaceutical agents. For example, the angiotensin therapeutic agent may be administered with albumin. The quantity of the additional pharmaceutical agent administered may vary depending on the cumulative therapeutic effect of the treatment including the angiotensin therapeutic agent and the additional pharmaceutical agent. For example, the quantity of albumin administered may be 1 gram of albumin per kilogram of body weight given intravenously on the first day, followed by 20 to 40 grams daily. Yet other additional pharmaceutical agents may be any one or more of midodrine, octreotide, somatostatin, vasopressin analog ornipressin, terlipressin, pentoxifylline, acetylcysteine, norepinephrine, misoprostol, etc. In some aspects, other natriuretic peptides may also be used in combination with the angiotensin therapeutic agent to remedy the impairment of sodium excretion associated with diseases discussed above. For example, natriuretic peptides may include any type of atrial natriuretic peptide (ANP), brain natriuretic peptide (BNP), C-type natriuretic peptide (CNP), and/or dendroaspis natriuretic peptide, etc. Several diuretic compounds may be used in combination with the angiotensin therapeutic agent to induce urine output. For example, any one or more of the xanthines such as caffeine, theophylline, theobromine; thiazides such as bendroflumethiazide, hydrochlorothiazide; potassium-sparing diuretics such as amiloride, spironolactone, triamterene, potassium canrenoate; osmotic diuretics such as glucose (especially in uncontrolled diabetes), mannitol; loop diuretics such as bumetanide, ethacrynic acid, furosemide, torsemide; carbonic anhydrase inhibitors such as acetazolamide, dorzolamide; Na—H exchanger antagonists such as dopamine; aquaretics such as goldenrod, juniper; arginine vasopressin receptor 2 antagonists such as amphotericin B, lithium citrate; acidifying salts such as CaCl₂, NH₄Cl; ethanol, water, etc. may be used in combination with the angiotensin therapeutic agent to treat the patient. The list of additional pharmaceutical agents described above is merely illustrative and may include any other pharmaceutical agents that may be useful for the treatment of hypotension and related conditions.

### Excipients

The pharmaceutical compositions of the present invention may also contain diluents, fillers, salts, buffers, stabilizers, solubilizers, and other materials well known in the art. The term “pharmaceutically acceptable carrier” refers to a non-toxic carrier that may be administered to a patient, together with a therapeutically effective substance (e.g., the angiotensin therapeutic agent, such as angiotensin II) of this invention, and which does not destroy the pharmacological activity of the therapeutically effective substance. The term “pharmaceutically acceptable” means a non-toxic material that does not interfere with the effectiveness of the biological activity of the active ingredient(s). The characteristics of the carrier will depend on the route of administration. The term “excipient” refers to an additive in a formulation or composition that is not a pharmaceutically active ingredient.

One of skill in the art would appreciate that the choice of any one excipient may influence the choice of any other excipient. For example, the choice of a particular excipient may preclude the use of one or more additional excipients because the combination of excipients would produce undesirable effects. One of skill in the art would be able to empirically determine which excipients, if any, to include in the compositions of the invention. Excipients of the invention may include, but are not limited to, co-solvents, solubilizing agents, buffers, pH adjusting agents, bulking agents, surfactants, encapsulating agents, tonicity-adjusting agents, stabilizing agents, protectants, and viscosity modifiers. In some aspects, it may be beneficial to include a pharmaceutically acceptable carrier in the compositions of the invention.

### Solubilizing Agents

In some aspects, it may be beneficial to include a solubilizing agent in the compositions of the invention. Solubilizing agents may be useful for increasing the solubility of any of the components of the formulation or composition, including an angiotensin therapeutic agent (e.g., angiotensin II) or an excipient. The solubilizing agents described herein are not intended to constitute an exhaustive list, but are provided merely as exemplary solubilizing agents that may be used in the compositions of the invention. In certain aspects, solubilizing agents include, but are not limited to, ethyl alcohol, tert-butyl alcohol, polyethylene glycol, glycerol, methylparaben, propylparaben, polyethylene glycol, polyvinyl pyrrolidone, and any pharmaceutically acceptable salts and/or combinations thereof.

pH-Adjusting Agents

In some aspects, it may be beneficial to adjust the pH of the compositions by including a pH-adjusting agent in the compositions of the invention. Modifying the pH of a formulation or composition may have beneficial effects on, for example, the stability or solubility of a therapeutically effective substance, or may be useful in making a formulation or composition suitable for parenteral administration. pH-adjusting agents are well known in the art. Accordingly, the pH-adjusting agents described herein are not intended to constitute an exhaustive list, but are provided merely as exemplary pH-adjusting agents that may be used in the compositions of the invention. pH-adjusting agents may include, for example, acids and bases. In some aspects, a pH-adjusting agent includes, but is not limited to, acetic acid, hydrochloric acid, phosphoric acid, sodium hydroxide, sodium carbonate, and combinations thereof.

The pH of the compositions of the invention may be any pH that provides desirable properties for the formulation or composition. Desirable properties may include, for example, stability, increased therapeutically effective substance retention as compared to compositions at other pHs, and improved filtration efficiency. In some aspects, the pH of the compositions of the invention may be from about 3.0 to about 9.0, e.g., from about 5.0 to about 7.0. In particular aspects, the pH of the compositions of the invention may be 5.5±0.1, 5.6±0.1, 5.7±0.1, 5.8±0.1, 5.9±0.1, 6.0±0.1, 6.1±0.1, 6.2±0.1, 6.3±0.1, 6.4±0.1, or 6.5±0.1.

### Buffers

In some aspects, it may be beneficial to buffer the pH by including one or more buffers in the compositions. In certain aspects, a buffer may have a pKa of, for example, about 5.5, about 6.0, or about 6.5. One of skill in the art would appreciate that an appropriate buffer may be chosen for inclusion in compositions of the invention based on its pKa and other properties. Buffers are well known in the art. Accordingly, the buffers described herein are not intended to constitute an exhaustive list, but are provided merely as exemplary buffers that may be used in the compositions of the invention. In certain aspects, a buffer may include one or more of the following: Tris, Tris HCl, potassium phosphate, sodium phosphate, sodium citrate, sodium ascorbate, combinations of sodium and potassium phosphate, Tris/Tris HCl, sodium bicarbonate, arginine phosphate, arginine hydrochloride, histidine hydrochloride, cacodylate, succinate, 2-(N-morpholino)ethanesulfonic acid (MES), maleate, bis-tris, phosphate, carbonate, and any pharmaceutically acceptable salts and/or combinations thereof.

### Surfactants

In some aspects, it may be beneficial to include a surfactant in the compositions of the invention. Surfactants, in general, decrease the surface tension of a liquid composition. This may provide beneficial properties such as improved ease of filtration. Surfactants also may act as emulsifying agents and/or solubilizing agents. Surfactants are well known in the art. Accordingly, the surfactants described herein are not intended to constitute an exhaustive list, but are provided merely as exemplary surfactants that may be used in the compositions of the invention. Surfactants that may be included include, but are not limited to, sorbitan esters such as polysorbates (e.g., polysorbate 20 and polysorbate 80), lipopolysaccharides, polyethylene glycols (e.g., PEG 400 and PEG 3000), poloxamers (i.e., pluronics), ethylene oxides and polyethylene oxides (e.g., Triton X-100), saponins, phospholipids (e.g., lecithin), and combinations thereof.

### Tonicity-Adjusting Agents

In some aspects, it may be beneficial to include a tonicity-adjusting agent in the compositions of the invention. The tonicity of a liquid composition is an important consideration when administering the composition to a patient, for example, by parenteral administration. Tonicity-adjusting agents, thus, may be used to help make a formulation or composition suitable for administration. Tonicity-adjusting agents are well known in the art. Accordingly, the tonicity-adjusting agents described herein are not intended to constitute an exhaustive list, but are provided merely as exemplary tonicity-adjusting agents that may be used in the compositions of the invention. Tonicity-adjusting agents may be ionic or non-ionic and include, but are not limited to, inorganic salts, amino acids, carbohydrates, sugars, sugar alcohols, and carbohydrates. Exemplary inorganic salts may include sodium chloride, potassium chloride, sodium sulfate, and potassium sulfate. An exemplary amino acid is glycine. Exemplary sugars may include sugar alcohols such as glycerol, propylene glycol, glucose, sucrose, lactose, and mannitol.

### Stabilizing Agents

In some aspects, it may be beneficial to include a stabilizing agent in the compositions of the invention. Stabilizing agents help increase the stability of a therapeutically effective substance in compositions of the invention. This may occur by, for example, reducing degradation or preventing aggregation of a therapeutically effective substance. Without wishing to be bound by theory, mechanisms for enhancing stability may include sequestration of the therapeutically effective substance from a solvent or inhibiting free radical oxidation of the anthracycline compound. Stabilizing agents are well known in the art. Accordingly, the stabilizing agents described herein are not intended to constitute an exhaustive list, but are provided merely as exemplary stabilizing agents that may be used in the compositions of the invention. Stabilizing agents may include, but are not limited to, emulsifiers and surfactants.

### Routes of Delivery

The compositions of the invention can be administered in a variety of conventional ways. In some aspects, the compositions of the invention are suitable for parenteral administration. These compositions may be administered, for example, intraperitoneally, intravenously, intrarenally, or intrathecally. In some aspects, the compositions of the invention are injected intravenously. One of skill in the art would appreciate that a method of administering a therapeutically effective substance formulation or composition of the invention would depend on factors such as the age, weight, and physical condition of the patient being treated, and the disease or condition being treated. The skilled worker would, thus, be able to select a method of administration optimal for a patient on a case-by-case basis.

Unless otherwise defined herein, scientific and technical terms used in this application shall have the meanings that are commonly understood by those of ordinary skill in the art. Generally, nomenclature and techniques relating to chemistry, molecular biology, cell and cancer biology, immunology, microbiology, pharmacology, and protein and nucleic acid chemistry, described herein, are those well-known and commonly used in the art.

Throughout this specification, the word “comprise” or variations such as “comprises” or “comprising” will be understood to imply the inclusion of a stated integer (or components) or group of integers (or components), but not the exclusion of any other integer (or components) or group of integers (or components). The singular forms “a,” “an,” and “the” include the plurals unless the context clearly dictates otherwise. The term “including” is used to mean “including but not limited to.” “Including” and “including but not limited to” are used interchangeably. The terms “patient” and “individual” are used interchangeably and refer to either a human or a non-human animal. These terms include mammals such as humans, primates, livestock animals (e.g., bovines, porcines), companion animals (e.g., canines, felines) and rodents (e.g., mice, rabbits and rats).

“About” and “approximately” shall generally mean an acceptable degree of error for the quantity measured given the nature or precision of the measurements. Typically, exemplary degrees of error are within 20%, preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

### INCORPORATION BY REFERENCE

All publications and patents mentioned herein are hereby incorporated by reference in their entirety as if each individual publication or patent was specifically and individually indicated to be incorporated by reference. In case of conflict, the present specification, including its specific definitions, will control. While specific aspects of the patient matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

1. A method of treating hypotension in a human patient receiving a vasopressor and having an initial mean arterial pressure, comprising:
administering to the patient a composition comprising an angiotensin therapeutic agent;
after a period of time, measuring the mean arterial pressure of the patient; and
if the measured mean arterial pressure is at or above 75 mm Hg, decreasing the rate at which the vasopressor is administered to the patient.

2. The method of claim 1, further comprising, if the measured mean arterial pressure is below 75 mm Hg, increasing the rate of administering the angiotensin therapeutic agent.

3. A method of treating hypotension in a human patient receiving a vasopressor and having an initial mean arterial pressure, comprising:
administering to the patient a composition comprising an angiotensin therapeutic agent;
after a period of time, measuring the mean arterial pressure of the patient; and
if the measured mean arterial pressure is at least 10 mm Hg higher than the initial mean arterial pressure, decreasing the rate at which the vasopressor is administered to the patient.

4. The method of claim 3, further comprising, if the measured mean arterial pressure is less than 5 mm Hg higher than the initial mean arterial pressure, increasing the rate of administering the angiotensin therapeutic agent.

5. The method of claim 1, wherein the vasopressor is a catecholamine, and the catecholamine is dopamine, norepinephrine, epinephrine, or phenylephrine.

6. The method of claim 5, wherein the patient is receiving at least 0.1 μg/kg/min of norepinephrine, at least 0.1 μg/kg/min of epinephrine, or at least 5 μg/kg/min of dopamine prior to administering the composition.

7. (canceled)

8. The method of claim 1, wherein the vasopressor is vasopressin or a vasopressin analog.

9-11. (canceled)

12. The method of claim 1, wherein the initial mean arterial pressure of the patient is 70 mm Hg or less.

13. The method of claim 12, wherein the initial mean arterial pressure of the patient is 65 mm Hg or less.

14-15. (canceled)

16. The method of claim 1, wherein the rate at which the vasopressor is administered is decreased to 0 ng/kg/min.

17. The method of claim 1, wherein the rate at which the vasopressor is administered is decreased by at least 60%.

18. The method of claim 1, wherein the angiotensin therapeutic agent is angiotensinogen, angiotensin I, angiotensin II, angiotensin III, or angiotensin IV.

19. The method of claim 18, wherein the angiotensin therapeutic agent is angiotensinogen, 1-L-aspartate-5-L-valine angiotensin I, 1-L-asparagine-5-L-valine angiotensin I, 1-L-asparagine-5-L-isoleucine angiotensin I, 1-L-aspartate-5-L-isoleucine angiotensin I, 1-L-aspartate-5-L-valine angiotensin II, 1-L-asparagine-5-L-valine angiotensin II, 1-L-asparagine-5-L-isoleucine angiotensin II, 1-L-aspartate-5-L-isoleucine angiotensin II, 4-L-valine angiotensin III, 4-L-isoleucine angiotensin III, 3-L-valine angiotensin IV, or 3-L-isoleucine angiotensin IV.

20-22. (canceled)

23. The method of claim 1, wherein the angiotensin therapeutic agent is administered at an initial rate of at least about 5 ng/kg/min.

24. The method of claim 1, wherein the angiotensin therapeutic agent is administered at an initial rate of at least about 10 ng/kg/min.

25-36. (canceled)

37. The method of claim 1, wherein the composition is administered until the mean arterial pressure of the patient can be maintained at or above 70 mm Hg with less than 0.1 μg/kg/min norepinephrine, less than 0.1 μg/kg/min epinephrine, less than 15 μg/kg/min dopamine, or less than 0.01 U/min vasopressin.

38-90. (canceled)

91. The method of claim 1, wherein administering comprises injection or intravenous infusion.

92. The method of claim 1, wherein the patient has a cardiovascular sequential organ failure assessment score (“SOFA score”) of 3 or 4.

93. The method of claim 92, wherein the patient has a cardiovascular SOFA score of 4.

94. The method of claim 1, wherein the patient has sepsis, septic shock, distributive shock, or cardiogenic shock.
